(19) United States
(12) Patent Application Publication
(10) Pub. No.: US 2010/0216885 A1
(43) Pub. Date: Aug. 26, 2010

(54) Orodispersible composition comprising polyunsaturated fatty acids without bad odor or taste

(76) Inventor: Catherine Kabaradjian, Vetraz-Mouthoux (FR)

Correspondence Address: BAYER HEALTHCARE LLC;CONSUMER CARE DIVISION, 36 COLUMBIA ROAD, MORRISTOWN, NJ, 07962, US

(21) Appl. No.: 12/799,743

(22) Filed: Apr. 30, 2010

(30) Foreign Application Priority Data
Oct. 30, 2007 (EP) 07291305.6

(51) Int. Cl. A61K 31/202

(52) U.S. Cl. 514/560

(57) ABSTRACT

An orodispersible composition without bad odor, smell, or taste comprising polyunsaturated fatty acids (PUFA) and a disintegrant. The disintegrant is a solid dispersion comprising mannitol, xylitol, microcrystalline cellulose, crospovidone and dibasic calcium phosphate. A process of preparing the composition and its use as nutritional or dietary supplement for balancing the blood lipid level, preventing or reducing the risk of the development of atherosclerotic changes, disorders or diseases.

### BACKGROUND OF THE INVENTION

1. Field of the Invention

This invention relates to an orodispersible composition comprising a polyunsaturated fatty acid and a disintegrant, its process for preparation and its use as a nutritional or dietary supplement for promoting balanced blood lipid levels and preventing or reducing the risk of the development of atherosclerotic changes, disorders or diseases. The composition does not have the unpleasant odor, smell, or taste often associated with other compositions containing polyunsaturated fatty acids.

2. Description of Related Art

Polyunsaturated fatty acids (“PUFAs”) are found in fish oil and may be responsible for reducing blood lipid levels. Lower blood lipid levels, in turn, may reduce hypertension as suggested by an epidemiological study carried out among the Inuits (M. H. Davidson, P. R. Liebson, “Marine Lipids and Atherosclerosis: A Review,” Cardiovascular Reviews & Reports, Vol. 7, No. 5, (1986)). In particular, the blood concentration of low density lipoprotein cholesterol (LDL) is lowered and the high density lipoprotein cholesterol (HDL) is increased among people consuming a diet with high levels of PUFAs. Coronary heart disease (CHD) is a major cause of death in the western countries, and high plasma cholesterol levels, especially when coupled with an unfavorable LDL/HDL ratio, is highly correlated with the risk of CHD (Willett, W. and Sacks, F., “Chewing the fat—how much and what kind?” N. Eng. J. Med., vol. 324(2) p. 121-23 (1991)).

The PUFAs found in fish oil have chains of 18, 20 or 22 carbon atoms and can be classified as n-3 omega and n-6 omega fatty acids, which are essential for the human body. In particular the omega-3 fatty acids eicosapentenoic acid (EPA) and docosahexenoic acid (DHA) are only found in fish and other marine life. Fish oil is therefore a very important food source for omega-3 fatty acids.

Standard nutritional supplement regimens that include supplements containing PUFAs generally call for a daily administration of from about 500 to about 1,000 mg of liquid fish oil. This amount of fish oil is normally contained in one or more (but usually one or two) capsules, tablets, or softgels. These dosage forms provide advantages over liquids because they use various technologies to limit the fishy smell and odor often associated with PUFAs.

Even with capsules, tablets or softgels, some people still experience gastrointestinal upset due to a perceived fishy smell, even hours after the fish oil has been consumed. One possible explanation for the source of this perceived fishy smell is that when, for example, a capsule containing fish oil dissolves in the gastro-intestinal tract, the entire voluminous dosage of the fish oil is released as a macroscopic drop that can delay and interfere with the absorption of the fish oil through the normal digestive process.

Various solutions to the problems posed by the fishy smell and odor and the delayed absorption of the PUFAs have been tried. Microdispersed fish oil preparations as a pulverulent or aqueous matrix, as described in European Patent No. 276,772 to Horn et al., published Jul. 15, 1992, were prepared by first preemulsifying a fish oil together with a surfactant, a protective colloid, and water in a conventional high-speed stirrer and then emulsifying the resulting mixture in a high pressure homogenizer to reduce the average diameter of the oil droplets below 10 μm. This formulation requires multiple extra processing steps if used in a commercial setting, and the microdispersed fish oil granules or powder can still have a fishy smell when pressed in simple tablets, so that flavors or antioxidants must be added to the formulations.

The fishy smell of the PUFAs appears to arise as a product of the oxidation of the unsaturated bonds in the PUFAs. Antioxidants, such as tocopherol, have been added to formulations containing PUFAs, which can prevent or delay oxidation and stabilize the PUFAs, as described in German Patent No. 20105126 to Bartz or European Patent No. 1,155,620 to Lystrup et al. (corresponding to U.S. Published Application No. US 2003/165596 to Lystrup, K., et al., published Sep. 4, 2003).

Some acceptable added flavors to mask the fishy smell of the fish oil are described in Japanese Patent Publication No. JP 08092587 (Shuichiro, U., et al., Published Apr. 9, 1996) or Japanese Patent Publication No. JP 08228678 (Yamashita, M., et al., Published Sep. 10, 1996). Taste masking may also be carried out by adding milk products to a fish oil formulation (as described in US Patent Publication No. 2003/0198728 to Sundram, K. et al, Published Oct. 23, 2003, and as described in Japanese Patent Publication No. JP 2002-204656, to Ogasawara, N., et al., published Jul. 23, 2002) or by coating or encapsulating the PUFAs (as described in PCT Publication No. WO 2004/016720 (also published as US Patent Publication No. 2006/068019 to Dalziel, S. et al., Published March 30, 2006) and WO 2005/029978 (also published as U.S. Patent Publication No.2007/031475 to Kuslys, M. et al., Published Feb. 8, 2007).

The reason for the fishy smell of the fish oil appears to be based on the oxidation of the unsaturated part of the PUFA. In order to prevent oxidation and to stabilize the PUFA antioxidants e.g. tocopherol can be added to the formulation as described e.g. in German Patent DE 20105126 or European Patent No. 1,155,620.

Despite attempts to mask the fishy odor that may come from oxidizing PUFAs and despite the efforts to delay or prevent the oxidization of the PUFAs, a need in the art remains for an oral composition providing the benefits of PUFAs without the unpleasant smell and taste associated with PUFAs.

### SUMMARY OF THE INVENTION

The object of the present invention is to provide an orodispersible composition containing PUFA, having no bad odor or smell and avoiding a complex and expensive taste masking technology.

Surprisingly it is found that a bad or fishy smell of the composition according to the present invention can be avoided.

### DESCRIPTION OF THE PREFERRED EMBODIMENTS

The invention comprises an orodispersible composition comprising at least one polyunsaturated fatty acid (PUFA) and a disintegrant. The disintegrant may be a solid dispersion comprising mannitol, xylitol, microcrystalline cellulose, crospovidone and dibasic calcium phosphate. The oral composition according to the invention does not have a fishy or bad smell, odor or taste.

Polyunsaturated fatty acids (PUFA) useful in the invention include, but are not limited to, fish oil, perilla oil, omega-3 fatty acids, omega-6-fatty acids, arachidonic acid, linoleic acid, alpha-linoleic acid, dihomogammalinoleic acid, eicosapentenoic acid (EPA), docosahexenoic acid (DHA) and mixtures thereof. Fish oil is preferred, as are omega-3 fatty acids, eicosapentenoic acid (EPA), docosahexenoic acid (DHA) and mixtures thereof Most preferred are fish oils containing eicosapentenoic acid (EPA) and docosahexenoic acid (DHA).

In a preferred embodiment, the composition comprises a tablet or other unit dosage form comprising at from about 5% to about 70% by weight of least one PUFA, preferably a fish oil powder or granulate. More preferably, the tablet or other unit dosage form comprises from about 40% to about 60% by weight of the PUFA. The tablet or other unit dosage form preferably comprises from about 100 mg to about 1,000 mg of PUFA, and more preferably from about 400 mg to about 900 mg.

In a highly preferred embodiment of the invention, the composition comprises from about 0.5% to about 10% eicosapentenoic acid (EPA), more preferably from about 1.5% to about 4% by weight of the composition. The eicosapentenoic acid (EPA) may also comprise from about 1% to about 20% by weight of a fish oil powder or granulate contained in the unit dosage form, and preferably from about 3% to about 8% by weight of the fish oil powder or granulate. The total amount of EPA in the tablet or other unit dosage form is preferably from about 10 mg to about 100 mg, and more preferably from about 15 mg to about 50 mg.

In a preferred embodiment of the invention, the composition comprises docosahexenoic acid (DHA) preferably from about 0.5% to about 10% by weight of the composition, and more preferably from about 1% to about 4% by weight of the composition. The DHA may also comprise from about 1% to about 20% by weight, preferably from about 2% to about 6% by weight of a fish oil powder or granulate contained in the composition. The total amount of DHA in a unit dose of the composition is preferably from about 10 mg to about 50 mg, and more preferably from about 15 mg to about 30 mg.

The PUFA, in particular the fish oil containing eicosapentenoic acid (EPA) and docosahexenoic acid (DHA), is used preferably in a microdispersed form as a granulate or powder, in a pulverulent or aqueous matrix as described in European Patent No. 276,772, incorporated herein by reference. Preference is given to a fish oil granulate or powder.

In a preferred embodiment the pulverulant matrix of the fish oil powder or granulate comprises at least a homogenous protective colloid, a surfactant, optionally a diluent, stabilizer and further pharmaceutical excipients. In the case of the aqueous matrix the mentioned ingredients are used as an aqueous solution.

The protective colloids of the pulverulant matrix include but are not limited to polypeptides such as gelatine, casein, caseinate, polysaccharides such as starch, dextrin, pectin, Arabic gum, milk, milk powder, polyvinyl alcohols, polyvinylpyrrolidone, vinylpyrrolidone-vinylacetate-copolymers, acrylic acid- and methacrylic acid-copolymers with acrylic acid- or methacrylic acid esters, methyl cellulose, carboxymethyl cellulose, hydroxypropyl cellulose, alginates or mixtures thereof.

Diluents of the pulverulant matrix include but are not limited to sugar or sugar alcohols e.g. saccharose, lactose, invert sugar, sorbit, mannit or glycerine.

Stabilizers of the pulverulant matrix include but are not limited to tocopherol, t-butyl hydroxytoluol, t-butyl hydroxyanisol and ethoxyquine.

Surfactants of the pulverulant matrix include but are not limited to esters of long chain fatty acids and ascorbic acid, esters of mono- and diglycerin and fatty acids and oxethylated derivatives thereof, esters of mono fatty acid glycerides with acetic acid, citric acid, lactic acid, diacetyltartrate, salts of 2-(2′-stearoyllactyl) lactic acid, polyglycerine fatty acid esters, sorbitan fatty acid esters, porpylenglycol-fatty acid esters, ascorbylpalmitate and lecithin.

The pulverulant matrix comprises from about 5% to about 70%, preferably from about 50% to about 60%, fish oil; from about 1% to about 30%, preferably from about 5% to about 15%, of one or more surfactants; from about 5% to about 50%, preferably from about 10% to about 40%, of a protective colloid; and from about 0% to about 70%, preferably from about 3% to about 35%, of a diluent, all measured as a weight percent of the dry mass of the fish oil powder or granulate.

In a preferred embodiment the pulverulant matrix comprises the fish oil in small particles having an average particle size of less than about 10 μm, more preferably less than about 1 μm, and most preferably less than about 0.5 μm.

The fish oil powder or granulate can be prepared as described in EP 276,772.

According to the present invention the solid dispersion responsible for rapid disintegration comprises mannitol, xylitol, microcrystalline cellulose, crospovidone and dibasic calcium phosphate wherein microcrystalline cellulose, crospovidone and dibasic calcium phosphate are dispersed in the mixture of mannitol and xylitol. The solid dispersion according to the invention can be prepared as described in EP 1,523,974, incorporated herein by reference.

The amount of mannitol and xylitol in the solid dispersion is from about 40% to about 90%, preferably from about 50% to about 80%, more preferably from about 60% to about 78%, and most preferably from about 62% to about 78% by weight of the solid dispersion, and the amount of mannitol and xylitol in the total composition is from about 18% to about 41%, preferably from about 23% to about 36%, more preferably from about 27% to about 35%, and most preferably from about 28% to about 35% by weight of the total composition.

The ratio by weight of mannitol and xylitol is from (98 to 67): (2 to 33), preferably from (98 to 87): (2 to 13), more preferably from (97 to 87): (3 to 13), and most preferably from (96 to 89): (4 to 11).

The amount of dibasic calcium phosphate (which corresponds to calcium monohydrogen phosphate) in the solid dispersion is from about 1% to about 30%, preferably about 2% to about 15%, and more preferably about 3% to about 8% by weight of the solid dispersion, and the amount of dibasic calcium phosphate in the total composition is from about 0.4% to about 13%, preferably about 1% to about 7%, and more preferably about 2% to about 4% by weight of the total composition.

The amount of microcrystalline cellulose in the solid dispersion is from about 8% to about 22%, preferably from about 10% to about 22%, more preferably from about 12% to about 21% by weight of the solid dispersion, and the amount of microcrystalline cellulose in the total composition is from about 3% to about 10%, preferably from about 4% to about 10%, more preferably from about 5% to about 9% by weight of the total composition.

The amount of crospovidone in the solid dispersion is from about 5% to about 15%, preferably from about 5% to about 14%, more preferably from about 6% to about 13% by weight of the solid dispersion, and the amount of crospovidone in the total composition is from about 2% to about 7%, preferably from about 2% to about 6%, more preferably from about 3% to about 6% by weight of the total composition.

In a preferred embodiment D-mannitol is used in the solid dispersion.

The composition according to the invention can comprise further active ingredients such as vitamins and minerals. Vitamins include, but are not limited to, vitamin A, beta carotene, vitamin C (ascorbic acid), vitamin D3 (cholecalcipherol), vitamin E (tocopherol acetate), vitamin B1 (thiamine), vitamin B2 (riboflavin), nicotinamide, vitamin B5 (panthothenic acid), vitamin B6 (pyridoxine), folic acid, vitamin B12 (cyanocobalamin), vitamin K1, vitamin K2, especially menaquinone 7-10, and biotin. Minerals include, but are not limited to, iron salts, copper salts, calcium salts such as calcium carbonate, calcium phosphate, calcium glycerophosphate; magnesium salts such as magnesium phosphate, magnesium sulphate (dihydrate) or magnesium oxide; zinc salts such as zinc citrate; selenium salts such as sodium selenate; potassium iodide; manganese salts such as manganese sulphate; molybdate salts such as sodium molybdate; chromium salts such as chromium chloride; sodium chloride and potassium chloride.

The composition according to the present invention can be used as nutritional supplement or as dietary supplement for balancing the blood lipid level, preventing or reducing the risk of the development of atherosclerotic changes, disorders or diseases in a patient. The inventive composition can also be used as nutritional or as dietary supplement for developing and maintaining the cognitive functions connected with e.g. eyes, memory, language etc. or for alleviating and/or preventing blood vessel diseases, cardiovascular, cerebrovascular and nervous diseases such as e.g. hypertension, cardiac infarction, Alzheimer, Parkinson and depression, or for alleviating hormonal, immunologic disorders or obesity. Furthermore, it can be used as nutritional or dietary supplement to support treatments of diabetes, cancer and/or inflammatory affections. A patient, for the purpose of this invention, is a mammal, including a human. The use as a nutritional or dietary supplement is especially preferred for pregnant women, children and elderly persons.

A further aspect of the invention is a method for balancing the blood lipid level, preventing or reducing the risk of the development of atherosclerotic changes, disorders or diseases, for developing and maintaining the cognitive functions connected with e.g. eyes, memory, language etc., for alleviating and/or preventing blood vessel diseases, cardiovascular, cerebrovascular and nervous diseases such as e.g. hypertension, cardiac infarction, Alzheimer, Parkinson and depression, or for alleviating hormonal, immunologic disorders or obesity or for supporting treatments of diabetes, cancer and/or inflammatory affections by administering the inventive composition as nutritional supplement or as dietary supplement to a patient which is, for the purpose of this invention, a mammal, including a human, especially pregnant women, children and elderly persons.

The composition according to the invention is administered orally one or more, preferably up to three, more preferably up to two times per day. With each administration the number of dosage forms taken in at the same time should not exceed two.

Nevertheless, it may in some cases be advantageous to deviate from the amounts specified, depending on body weight, individual behavior toward the active ingredient, type of preparation and time or interval over which the administration is effected. For instance, less than the aforementioned minimum amounts may be sufficient in some cases, while the upper limit specified has to be exceeded in other cases.

Ingredients of the oral dosage form are those which are accepted for pharmaceuticals and nutritional supplements and physiologically unobjectionable, for example: as fillers cellulose derivatives (e.g. microcrystalline cellulose), sugars (e.g. lactose), sugar alcohols (e.g. mannitol, sorbitol), inorganic fillers (e.g. calcium phosphates), binders (e.g. polyvinylpyrrolidone, gelatin, starch derivatives and cellulose derivatives), and all other excipients required to produce formulations of pharmaceuticals and nutritional supplements of the desired properties, e.g. lubricants (magnesium stearate), e.g. disintegrants (e.g. crosslinked polyvinylpyrrolidone, sodium carboxymethylcellulose), e.g. wetting agents (e.g. sodium lauryl sulphate), e.g. release-slowing agents (e.g. cellulose derivatives, polyacrylic acid derivatives), e.g. coloured pigments.

Excipients for pharmaceuticals and nutritional supplements familiar to the skilled person are also described for example in the following handbook: “Handbook of Pharmaceutical Excipients”, Rowe R. C., Sheskey P. J. & Weller, P. J., American Pharmaceutical Association, Washington, 4th edition 2003.

The orodispersible tablet of the invention may be produced by known processes. The solid dispersion may be produced as described in European Patent No. 1,523,974. Tablets can be produced by mixing and/or granulating the active ingredients together with the excipients (e.g. the whole solid dispersion part) to form a blend which is finally pressed to tablets. Optionally, different blends containing different ingredients and excipients can be premixed and combined to a final blend which is then pressed to tablets.

An advantage of the composition of the present invention is that for the preparation of the composition a complex and expensive taste masking technology known in the prior art such as coating of tablets or granules, adding of antioxidants, or putting the PUPA into a capsule is not needed. The composition of the present invention can be prepared by simple and well-known standard procedures. Another well-known taste masking method is the addition of flavors in order to cover and mask the bad smell. This taste masking method is normally restricted to only a few applicable flavors which have to be selected in each case. However, flavoring ingredients are not needed for taste masking in the composition of the present invention.

Preference is given to an orodispersible composition which is not a coated tablet, coated granule, or capsule or which does not comprise an antioxidant or flavor or other taste masking substance.

Preference is given to a fast disintegrating orodispersible tablet, that means it disintegrates rapidly in the oral cavity. The disintegration time of the fast disintegrating orodispersible tablet may be equal to or less than about 100 seconds, preferably equal to or less than about 80 seconds.

The composition according to the present invention shows a good and/or fast absorption of the PUFA after administration of the composition. Furthermore the composition facilitates a quick intake, optionally without water or other drink.

The composition according to the invention shows an acceptable hardness and friability to be manufactured without affecting the beadlet integrity, i.e. no PUFA exudates from the tablet matrix.

### Example 1

An orodispersible tablet was prepared comprising: 500 mg of dry fish oil powder (omega-3, 18:12) including 23.5 mg of EPA and 16.5 mg of DHA; 500 mg of F-melt type C; and optionally the following excipients: 15 mg of anhydrous citric acid, and 10 mg of Aspartame.

F-melt type C is a commercially available disintegrant and comprises crospovidone, mannitol, xylitol, dibasic calcium phosphate and microcrystalline cellulose (Fuji, Chemical Industry Co., Ltd., Japan) and can be prepared as described in European Patent No. 1,523,974.

### Example 2

An orodispersible tablet was prepared comprising: 890 mg of dry fish oil powder (omega-3, 18:12) including 41.83 mg of EPA and 29.37 mg of DHA; 750 mg of F-melt type C; and optionally the following flavors and excipients: 25 mg of anhydrous citric acid, 35 mg of Aspartame, 5 mg of stearic acid, and 5 mg of iron oxide (red).

F-melt type C is a commercially available disintegrant and consists of crospovidone, mannitol, xylitol, dibasic calcium phosphate and microcrystalline cellulose (Fuji, Chemical Industry Co., Ltd., Japan) and can be prepared as described in European Patent No. 1,523,974.

### Example 3

An orodispersible tablet comprising: 500 mg of dry fish oil powder (omega-3, 18:12) including 23.5 mg of EPA and 16.5 mg of DHA; 500 mg of F-melt type C; and optionally the following flavors and excipients: 15 mg of anhydrous citric acid, 50 mg of Lemon flavor, 20 mg of honey flavor, and 10 mg of Aspartame.

F-melt type C is a commercially available disintegrant and consists of crospovidone, mannitol, xylitol, dibasic calcium phosphate and microcrystalline cellulose (Fuji, Chemical Industry Co., Ltd., Japan) and can be prepared as described in European Patent No. 1,523,974.

### Example 4

An orodispersible tablet comprising: 890 mg of dry fish oil powder (omega-3, 18:12) including 41.83 mg of EPA and 29.37 mg of DHA; 750 mg of F-melt type C; and optionally the following flavors and excipients: 25 mg of anhydrous citric acid, 50 mg of Lemon flavor, 20 mg of honey flavor, 30 mg of pineapple flavor, 35 mg of Aspartame, 5 mg of stearic acid, and 5 mg of iron oxide (red).

F-melt type C is a commercially available disintegrant and consists of crospovidone, mannitol, xylitol, dibasic calcium phosphate and microcrystalline cellulose (Fuji, Chemical Industry Co., Ltd., Japan) and can be prepared as described in European Patent No. 1,523,974.

The compositions were manufactured using a direct compression process. All the ingredients are mixed together in a tumble mixer for 20 min. and optionally 5 min. before the end the lubricant, if any, (stearic acid) is added. The final blend is pressed into tablets with a rotary press.

Examples 1 and 2 do not show any bad or fishy taste or smell, in examples 3 and 4 only small amounts of flavor are added to improve the taste of the otherwise savorless tablet. The disintegration time is 40 ±5 sec. for examples 1 and 3 and 60±5 sec. for examples 2 and 4.

What is claimed is:

1. An Orodispersible composition comprising:
(a) at least one polyunsaturated fatty acid; and
(b) a disintegrant comprising a solid dispersion comprising mannitol, xylitol, microcrystalline cellulose, crospovidone and dibasic calcium phosphate.

2. The composition of claim 1, wherein said at least one polyunsaturated fatty acid is selected from the group consisting of fish oil, perilla oil, omega-3 fatty acids, omega-6-fatty acids, arachidonic acid, linoleic acid, alpha-linoleic acid, dihomogammalinoleic acid, eicosapentenoic acid (EPA), docosahexenoic acid (DHA) or mixtures thereof.

3. The composition of claim 2, wherein said at least one polyunsaturated fatty acid comprises fish oil comprising eicosapentenoic acid (EPA) and docosahexenoic acid (DHA).

4. The composition of claim 3, wherein said at least one polyunsaturated fatty acid comprises a fish oil powder or granulate.

5. The composition of claim 1, wherein said solid dispersion comprises microcrystalline cellulose, crospovidone and dibasic calcium phosphate dispersed in a mixture of mannitol and xylitol.

6. The composition of claim 1, further comprising at least one mineral or vitamin.

7. The composition of claims 1, in the form of a fast disintegrating tablet having a disintegration time of 100 seconds or less.

8. The use of the composition of claim 1 for treatment of a human to accomplish at least one benefit selected from the group consisting of balancing the blood lipid level, preventing or reducing the risk of the development of atherosclerotic changes, disorders or diseases, developing or maintaining cognitive functions, alleviating or preventing blood vessel diseases, cardiovascular diseases, cerebrovascular diseases or nervous diseases, alleviating hormonal disorders, immunologic disorders or obesity, supporting treatments of diabetes, cancer or inflammatory afflictions.
